(19) United States
(12) Patent Application Publication
(10) Pub. No.: US 2005/0008647 A1
(43) Pub. Date: Jan. 13, 2005

(54) Cancer metastasis interfering vaccine

(76) Inventor: George Koliakos, Thessaloniki (GR)

Correspondence Address: KOLIAKOS GEORGE, P.O.B. 17034, THESSALONIKI, 542 10, GR

(21) Appl. No.: 10/706,406

(22) Filed: Oct. 31, 2003

(30) Foreign Application Priority Data
May 9, 2001 (GR) 20010100237

(51) Int. Cl. A61K 39/395

(52) U.S. Cl. 424/178.1

(57) ABSTRACT

A vaccine that has been synthesized by a polyvalent substrate and the idiotype parts of antibodies that recognize antimetastatic peptides. The vaccine induces the production of antiidiotype antibodies that inhibit metastasis formation. It is known that the ability of a cancer cell to metastasize is primary dependent by its property to bind with extracellular matrix proteins such as laminin and fibronectin. On these proteins the antimetastatic sequences (peptides) YIGSR and RGD have been discovered since 1991. Attempts to use these peptides either per se or as constituents of polymers in order to inhibit spontaneous metastasis in experimental animals had only limited results because of the limited life time of these preparation in the circulation. Using the described vaccine the immune system is leaded to the production of antiidiotype antibodies that have shape and properties similar to the antimetastatic peptides YIGSR and RGD. Pilot experiments indicated that these antibodies can inhibit the formation of spontaneous metastasis in experimental animals.

The present invention report describes a novel molecule that has been synthesized by a multivalent substrate and the idiotype parts of antibodies that recognize and bind antimetastatic peptides.

The molecule is used for the production by the living organism of antiidiotype antibodies¹⁻⁴ with antimetastatic properties.

The ability of cancer to metastasize is the most lethal characteristic of this disease⁵, that remains mainly incurable and one of the most common causes of mortality in well developed countries⁶. Therefore the suppression and eradication of metastases is a major goal of alternative treatment strategies for cancer⁶.

The ability of a cancer cell to metastasize depends by several properties, however high affinity binding with extracellular matrix molecules is currently considered as necessary.^{7 8-12}.

This ability is attributed to the enrichment of specific metastatic cell surface binding sites. These binding sites recognize specific aminoacid sequences situated on some extracellular matrix proteins such as laminin and fibronectin¹³.

It has been reported that proteolytic fragments of laminin and fibronectin occupy metastatic cell surface binding sites and thus inhibit experimental metastasis¹⁴. The aminoacid sequences that are immediately related with this property have been discovered and described^{10 15-29}. The sequence (peptide) YIGSR that recognizes a metastasis associated high affinity laminin receptor has been discovered on laminin³⁰ and the sequence RGDS, that recognizes a family of extracellular matrix receptors called integrins has been discovered on fibronectin^{23-26,31-34 35,36}.

It is presently well known that the metastatic ability of cancer cells can be experimentally inhibited if the binding sites described above are covered by the synthetic peptide YIGSR³⁷. The same peptide has been also used for the in vitro selection of melanoma cell lines with high metastatic potential³⁸. Moreover the radiolabeled peptides YIGSR and RGDS have been used in vivo for the detection of metastatic sites^{28,29 39}.

It was thus obvious since 1991⁴⁰ that polypeptides which contain RGD and/or YIGSR sequences could provide a promising approach for the control and prevention of cancer metastasis.

However, despite of the above evidence, the peptides RGD or YIGSR, had no effect in the spontaneous metastasis model, and only multiple intravenous administrations of polymers containing these sequences may result in a reduction of metastatic sites⁴⁰.

Several polymers containing these sequences have been proposed as antimetastatic agents in the past^{35,40,41,41-58}. Unfortunately these efforts had minimum success because of the limited life span of these molecules in plasma^{47,57,59-61}.

The advantage of the present invention is that after vaccination with the molecule we describe, the immunological system is directed to produce specific antibodies with idiotype sequences that are similar to the antimetastatic peptides. Thus the living organism produces antibodies with properties comparable with the described peptide properties. As our pilot experiments indicated these antibodies offer significant defense against cancer metastasis.

During these experiments, serum derived from rabbits immunized with the peptide YIGSR has been used for the vaccination of mice, while other mice (controls) were immunized with non specific rabbit serum. After vaccination all mice were inoculated with the same amount of Lewis lung carcinoma cells 3LL. The mice were sacrificed after 20 days and the lungs were observed for the evidence of metastasis macroscopically and microscopically.

The immunized mice had significantly smaller tumors and less micrometastases around the lung vessels in comparison to controls. Macroscopically lung metastases were obvious only in control mice.

According to the present innovation for the preparation of the vaccine molecules of polylysine, polyethylenoglycol or any other polyvalent molecule can be used. On these molecules multiple Fab fragments or V regions (idiotypes) of gamma globulins (antibodies) are covalently attached. These parts have been prepared by polyclonal or monoclonal antibodies or by bio-engineering methods. The antibodies have been raised against the antimetastatic peptides YIGSR and/or RGD or other molecules containing these sequences. Thus novel polyvalent antigenic molecules are synthesized that can be used as antigens (vaccines). The multiple antigen recognition sequences (idiotypes) that are included in this molecule, have been raised against antimetastatic peptides YIGSR and/or RGD and for this reason have a shape complimentary to these peptides. Thus this molecule will lead the immune system to produce antiidiotype antibodies with a shape and properties that are analog to the original molecules (peptides).

### Reference List

1. Pimm,M. V. & Gribben,S. J. Toxicity associated with the formation and clearance of immune complexes between antitumour monoclonal antibodies and syngeneic anti-idiotypic antibodies in mice. J. Cancer Res. Clin. Oncol. 119, 41-45 (1992).

2. Pimm,M. V. & Gribben,S. J. Influence of syngeneic (anti-idiotypic) antibody responses on biodistribution and tumour localisation of murine monoclonal antibodies and fragments. Anticancer Res. 13, 241-248 (1993).

3. Kairemo,K. J. Radioimmunotherapy of solid cancers: A review. Acta Oncol. 35, 343-355 (1996).

4. Fehr,T. et al. Role of repetitive antigen patterns for induction of antibodies against antibodies. J. Exp. Med. 185, 1785-1792 (1997).

5. Hansen,J. Common cancers in the elderly. Drugs Aging 13, 467-478 (1998).

6. Lode,H. N. et al. Synergy between an antiangiogenic integrin alphav antagonist and an antibody-cytokine fusion protein eradicates spontaneous tumor metastases. Proc. Natl. Acad. Sci. U.S.A. 96, 1591-1596 (1999).

7. Kramer,R. H., Gonzalez,R. & Nicolson,G. L. Metastatic tumor cells adhere preferentially to the extracellular matrix underlying vascular endothelial cells. Int. J. Cancer 26, 639-645 (1980).

8. Buck,C. A. & Horwitz,A. F. Cell surface receptors for extracellular matrix molecules. Annu. Rev. Cell Biol. 3, 179-205 (1987).

9. Buck,C. A. & Horwitz,A. F. Integrin, a transmembrane glycoprotein complex mediating cell-substratum adhesion. J. Cell Sci. Suppl 8, 231-250 (1987).

10. Fields,G. B. Synthetic peptides and tumor cell metastasis. Pept. Res. 6, 115-120 (1993).

11. Lochter,A. & Bissell,M. J. Involvement of extracellular matrix constituents in breast cancer. Semin. Cancer Biol. 6, 165-173 (1995).

12. Humphries,M. J., Yasuda,Y., Olden,K. & Yamada,K. M. The cell interaction sites of fibronectin in tumour metastasis. Ciba Found. Symp. 141, 75-93 (1988).

13. Castronovo,V. & Sobel,M. E. Laminin and fibronectin increase the steady state level of the 67 kD high affinity metastasis-associated laminin receptor mRNA in human cancer cells. Biochem. Biophys. Res. Commun. 168, 1110-1117 (1990).

14. McCarthy,J. B., Skubitz,A. P., Palm,S. L. & Furcht,L. T. Metastasis inhibition of different tumor types by purified laminin fragments and a heparin-binding fragment of fibronectin. J. Natl. Cancer Inst. 80, 108-116 (1988).

15. Castronovo,V. et al. Immunodetection of the metastasis-associated laminin receptor in human breast cancer cells obtained by fine-needle aspiration biopsy. Am. J. Pathol. 137, 1373-1381 (1990).

16. Cioce,V. et al. Increased expression of the laminin receptor in human colon cancer. J. Natl. Cancer Inst. 83, 29-36 (1991).

17. Castronovo,V. Laminin receptors and laminin-binding proteins during tumor invasion and metastasis. Invasion Metastasis 13, 1-30 (1993).

18. Cioce,V., Margulies,I. M., Sobel,M. E. & Castronovo,V. Interaction between the 67 kilodalton metastasis-associated laminin receptor and laminin. Kidney Int. 43, 30-37 (1993).

19. Magnifico,A. et al. Peptide G, containing the binding site of the 67-kDa laminin receptor, increases and stabilizes laminin binding to cancer cells. J. Biol. Chem. 271, 31179-31184 (1996).

20. Waltregny,D., de Leval,L., Menard,S., de Leval,J. & Castronovo,V. Independent prognostic value of the 67-kd laminin receptor in human prostate cancer. J. Natl. Cancer Inst. 89, 1224-1227 (1997).

21. Menard,S., Castronovo,V., Tagliabue,E. & Sobel,M. E. New insights into the metastasis-associated 67 kD laminin receptor. J. Cell Biochem. 67, 155-165 (1997).

22. Alino,S. F., Unda,F. J. & Perez-Yarza,G. Laminin surface binding sites and metastatic potential of 3LL tumor cells, increased by indomethacin. Biochem. Biophys. Res. Commun. 167, 731-738 (1990).

23. Ruoslahti,E. & Giancotti,F. G. Integrins and tumor cell dissemination. Cancer Cells 1, 119-126 (1989).

24. Ruoslahti,E. How cancer spreads. Sci. Am. 275, 72-77 (1996).

25. Ruoslahti,E. Integrins as signaling molecules and targets for tumor therapy. Kidney Int. 51, 1413-1417 (1997).

26. Yi,M. & Ruoslahti,E. A fibronectin fragment inhibits tumor growth, angiogenesis, and metastasis. Proc. Natl. Acad Sci. U.S.A. 98, 620-624 (2001).

27. Koliakos,G. G., Tsilibary,E. C. & Charonis,A. S. A melanoma cell surface laminin binding protein with apparent Mr 90,000. Connect. Tissue Res. 26, 167-180 (1991).

28. Kouzi-Koliakos,K. et al. In vivo binding of the radioiodinated peptide YIGSR on B16 melanoma cells. Invasion Metastasis 16, 322-329 (1996).

29. Koliakos,G., Trontzos,C., Kouzi-Koliakos,K., Kanellaki,M. & Grammaticos,P. Lung carcinoma imaging using a synthetic laminin derivative radioiodinated peptide YIGSR. J. Nucl. Med. 38, 1940-1944 (1997).

30. Iwamoto,Y. et al. Inhibition of angiogenesis, tumour growth and experimental metastasis of human fibrosarcoma cells HT1080 by a multimeric form of the laminin sequence Tyr-Ile-Gly-Ser-Arg (YIGSR). Br. J. Cancer 73, 589-595 (1996).

31. Ruoslahti,E. Cell adhesion and tumor metastasis. Princess Takamatsu Symp. 24, 99-105 (1994).

32. Ruoslahti,E. Fibronectin and its alpha 5 beta 1 integrin receptor in malignancy. Invasion Metastasis 14, 87-97 (1994).

33. Pasqualini,R., Bourdoulous,S., Koivunen,E., Woods,V. L. & Ruoslahti,E. A polymeric form of fibronectin has antimetastatic effects against multiple tumor types. Nat. Med. 2, 1197-1203 (1996).

34. Ruoslahti,E. Fibronectin and its integrin receptors in cancer. Adv. Cancer Res. 76, 1-20 (1999).

35. Iwamoto,Y. et al. YIGSR, a synthetic laminin pentapeptide, inhibits experimental metastasis formation. Science 238, 1132-1134 (1987).

36. Graf,J. et al. A pentapeptide from the laminin B1 chain mediates cell adhesion and binds the 67,000 laminin receptor. Biochemistry 26, 6896-6900 (1987).

37. Alino,S. F., Unda,F. J. & Perez-Yarza,G. Laminin surface binding sites and metastatic potential of 3LL tumor cells, increased by indomethacin. Biochem. Biophys. Res. Commun. 167, 731-738 (1990).

38. Yamamura,K., Kibbey,M. C. & Kleinman,H. K. Melanoma cells selected for adhesion to laminin peptides have different malignant properties. Cancer Res. 53, 423-428 (1993).

39. Sivolapenko,G. B. et al. Imaging of metastatic melanoma utilising a technetium-99 m labelled RGD-containing synthetic peptide. Eur. J Nucl. Med. 25, 1383-1389 (1998).

40. Soszka,T. et al. Inhibition of murine melanoma cell-matrix adhesion and experimental metastasis by albolabrin, an RGD-containing peptide isolated from the venom of Trimeresurus albolabris. Exp. Cell Res. 196, 6-12 (1991).

41. Saiki,I. et al. Antimetastatic effects of synthetic polypeptides containing repeated structures of the cell adhesive Arg-Gly-Asp (RGD) and Tyr-Ile-Gly-Ser-Arg (YIGSR) sequences. Br. J. Cancer 60, 722-728 (1989).

42. Saiki,I. et al. Anti-metastatic and anti-invasive effects of polymeric Arg-Gly-Asp (RGD) peptide, poly(RGD), and its analogues. Jpn. J. Cancer Res. 81, 660-667 (1990).

43. Kumagai,H., Tajima,M., Ueno,Y., Giga-Hama,Y. & Ohba,M. Effect of cyclic RGD peptide on cell adhesion and tumor metastasis. Biochem. Biophys. Res. Commun. 177, 74-82 (1991).

44. Komazawa,H. et al. Inhibition of tumor metastasis by Arg-Gly-Asp-Ser (RGDS) peptide conjugated with sulfated chitin derivative, SCM-chitin-RGDS. Clin. Exp. Metastasis 11, 482-491 (1993).

45. Hyacinthe,L. M., Jarrett,T. W., Gordon,C. S., Vaughan,E. D. & Whalen,G. F. Inhibition of bladder tumor cell implantation in cauterized urothelium, without inhibition of healing, by a fibronectin-related peptide (GRGDS). Ann. Surg. Oncol. 2, 450-456 (1995).

46. Fujii,H. et al. Antimetastatic activities of synthetic Arg-Gly-Asp-Ser (RGDS) and Arg-Leu-Asp-Ser (RLDS) peptide analogues and their inhibitory mechanisms. Biol. Pharm. Bull. 18, 1681-1688 (1995).

47. Fujii,H. et al. Inhibition of tumor invasion and metastasis by peptidic mimetics of Arg-Gly Asp (RGD) derived from the cell recognition site of fibronectin. Oncol. Res. 8, 333-342 (1996).

48. Miyata,K. et al. A YIGSR-containing novel mutein without the detrimental effect of human TNF-alpha of enhancing experimental pulmonary metastasis. Clin. Exp. Metastasis 10, 267-272 (1992).

49. Yamamura,K., Kibbey,M. C., Jun,S. H. & Kleinman,H. K. Effect of Matrigel and laminin peptide YIGSR on tumor growth and metastasis. Semin. Cancer Biol. 4, 259-265 (1993).

50. Nomizu,M., Yamamura,K., Kleinman,H. K. & Yamada,Y. Multimeric forms of Tyr-Ile-Gly-Ser-Arg (YIGSR) peptide enhance the inhibition of tumor growth and metastasis. Cancer Res. 53, 3459-3461 (1993).

51. Kim,W. H., Schnaper,H. W., Nomizu,M., Yamada,Y. & Kleinman,H. K. Apoptosis in human fibrosarcoma cells is induced by a multimeric synthetic Tyr-Ile-Gly-Ser-Arg (YIGSR)-containing polypeptide from laminin. Cancer Res. 54, 5005-5010 (1994).

52. Kaneda,Y. et al. Synthetic cell-adhesive laminin peptide YIGSR conjugated with polyethylene glycol has improved antimetastatic activity due to a longer half-life in blood. Invasion Metastasis 15, 156-162 (1995).

53. Zalipsky,S., Puntambekar,B., Boulikas,P., Engbers,C. M. & Woodle,M. C. Peptide attachment to extremities of liposomal surface grafted PEG chains: preparation of the long-circulating form of laminin pentapeptide, YIGSR. Bioconjug. Chem. 6, 705-708 (1995).

54. Sivanandaiah,K. M. et al. Synthetic peptides related to laminin pentapeptide (YIGSR) fragment. Indian J. Exp. Biol. 34, 658-662 (1996).

55. Zhao,M., Kleinman,H. K. & MokotoffM. Synthesis and activity of partial retro-inverso analogs of the antimetastatic laminin-derived peptide, YIGSR-NH2. J Pept. Res. 49, 240-253 (1997).

56. Maeda,M. et al. Amino acids and peptides. XXXI. Preparation of analogs of the laminin-related peptide YIGSR and their inhibitory effect on experimental metastasis. Chem. Pharm. Bull. (Tokyo) 46, 347-350 (1998).

57. Mu,Y. et al. Bioconjugation of laminin peptide YIGSR with poly(styrene co-maleic acid) increases its antimetastatic effect on lung metastasis of B16-BL6 melanoma cells. Biochem. Biophys. Res. Commun. 255, 75-79 (1999).

58. Mu,Y. et al. Bioconjugation of laminin-related peptide YIGSR with polyvinyl pyrrolidone increases its antimetastatic effect due to a longer plasma half-life. Biochem. Biophys. Res. Commun. 264, 763-767 (1999).

59. Zamora,P. O., Eshima,D., Graham,D., Shattuck,L. & Rhodes,B. A. Biological distribution of 99 mTc-labeled YIGSR and IKVAV laminin peptides in rodents: 99 mTc-IKVAV peptide localizes to the lung. Biochim. Biophys. Acta 1182, 197-204 (1993).

60. Fujii,H. et al. A new pseudo-peptide of Arg-Gly-Asp (RGD) with inhibitory effect on tumor metastasis and enzymatic degradation of extracellular matrix. Clin. Exp. Metastasis 16, 94-104 (1998).

61. Ohnishi,Y. et al. A new pseudo-peptide analogue of the Arg-Gly-Asp (RGD) sequence inhibits liver metastasis of colon 26-L5 carcinoma cells. Cancer Lett. 124, 157-163 (1998).

1. A vaccine that is composed by a polyvalent substrate (polylysine, polyethylenglucole, or any other polyvalent molecule). On this molecule multiple Fab parts or V regions (idiotypes) of gamma globulins (antibodies) have been covalently bound. These parts have been prepared by polyclonal or monoclonal antibodies or by other biotechnological or by direct biochemical synthesis. These antibodies have been raised against synthetic peptides with known antimetastatic properties.
These molecules are used alone or in combination with other molecules for the vaccination of humans or other living organisms aiming to inhibit the formation of cancer metastasis.

2. A vaccine according to the claim 1, that is not composed by a polyvalent substrate. Instead the multiple Fab parts or V regions of gamma globulins are directly bound to each other. By this way the composed molecule consists by multiple parts or multiple synthetic analogs of Fab parts or V regions of gamma globulins.
